(19) United States
(12) Plant Patent Application Publication
(10) Pub. No.: US 2018/0213707 P1
(43) Pub. Date: Jul. 26, 2018

(54) X HEURECHELLA PLANT NAMED 'GALACTICA'

(50) ;

(71) Applicant: Hans A. Hansen, Zeeland, MI (US)

(72) Inventor: Hans A. Hansen, Zeeland, MI (US)

(73) Assignee: Walters Gardens, Inc., Zeeland, MI (US)

(21) Appl. No.: 15/530,534

(22) Filed: Jan. 25, 2017

(51) Int. Cl. A01H 6/80

(52) U.S. Cl. PLT/441

(57) ABSTRACT

The new hybrid X Heucherella plant named ‘Galactica’ with deeply-incised, palmately lobed foliage of variable coloration starting in the spring as coppery-red with burgundy veins, transitioning to deep burgundy with silvery-gray surrounding veins during flowering and in winter becoming deep green with silver frosting and black mahogany surrounding veins. ‘Galactica’ begins flowering in late spring with a near white with slight blush of pink. The new plant is suitable for landscaping en masse, as a specimen accent or as a container plant.

Botanical denomination: X Heucherella (Heuchera x Tiarella).

Cultivar designation: ‘Galactica’.

### BACKGROUND OF THE INVENTION

The present invention relates to a new and distinct intergeneric hybrid between coral bells and foam flower, both in the Saxifragaceae family and given the cultivar name of ‘Galactica’ with the combined generic epithet X Heucherella. The intergeneric hybrid is sometimes given the common name of foamy bells. X Heucherella ‘Galactica’ resulted from an intentional cross between the unreleased proprietary Heuchera hybrid known by the breeder code K10-70-69 (not patented) as the female or seed parent and Tiarella ‘Jade Peacock’ U.S. Plant Pat. No. 26,730 as the male or pollen parent. The new plant was hybridized by the inventor at a wholesale perennial nursery in Zeeland, Mich., USA on Feb. 13, 2013 and harvested in the spring of 2013. The new plant passed the original evaluation in summer 2013 and was given the initial breeder code of 13-202-4 through the remaining trial period. ‘Galactica’ was selected from among many other crosses and X Heucherella seedlings growing at the same nursery in Zeeland, Mich. which met the rigorous criteria of excellent foliage, flower and habit established as breeding goals. X Heucherella ‘Galactica’ has been asexually propagated since 2014 by basal cuttings at a nursery in Zeeland, Mich. and also by careful shoot-tip tissue culture propagation. The resultant asexually propagated plants have remained stable and exhibit the same characteristics as the original plant.

No plants of X Heucherella ‘Galactica’ have been sold, under this or any name, in this country or anywhere in the world, prior to the filing of this application, nor has any disclosure of the new plant been made prior to the filing of this application with the exception of that which may have been disclosed or sold within one year of the filing of this application and was either derived directly or indirectly from the inventor.

### BRIEF SUMMARY OF THE INVENTION

‘Galactica’ differs from its parents as well as all other Tiarella, Heuchera or X Heucherella known to the applicant. Compared with the female parent, the new plant has whiter flowers with a blush of pink, the foliage more deeply dissected with longer lobes, and the foliage color has deeper green coloration in the summer. Compared to the male parent, the new plant has more burgundy foliage earlier in the spring with less dissected lobes, the flowers are more white with a blush of pink rather than creamy-white and the scapes are taller and more branched in the lower portion. The most similar cultivars include: X Heucherella ‘Twilight’ U.S. Plant Pat. No. 25,723, ‘Tapestry’ U.S. Plant Pat. No. 21,150 and ‘Cracked Ice’ U.S. Plant Pat. No. 24,690.

‘Twilight’ has less cleft foliage with more acute lobe apices, and the leaf color is more gray on top with less contrasting deep green, and a lighter wine color underneath than ‘Galactica’. ‘Tapestry’ has more broadly divergent dissected lobes, the foliage coloration does not have the silvery gray between the veins along with the broadly diffused burgundy along the leaf veins, and the flower color is pink rather than white with a blush of pink. The new plant has foliage that is more deeply cleft with more acute and less rounded indentations and the underside of the foliage is less maroon-colored than ‘Cracked Ice’.

The new plant differs from all Heuchera, X Heucherella and Tiarella known to the inventor in the following combined traits:
1. The foliage color is variable with the seasons and development of the leaves.
2. Leaves emerge in the spring with coppery-red with burgundy red veins.
3. Leaves transition to deep burgundy surrounding the veins and silvery-gray contrast between the veins.
4. Leaves develop to a deep green with silver frosting between the veins and a deep black-mahogany surrounding the veins.
5. The flowers open near-white with a slight pink blush from pink buds.
6. Foliage is deeply-incised with narrow separation between broad, frequently slightly overlapping lobes.
7. Habit is vigorous and mounded with multiple tightly clustered shoots emerging at the base all season.
8. The plant is robust, seedless, compact and is more heat and sun tolerant than typical X Heucherella.

### BRIEF DESCRIPTION OF THE DRAWINGS

The photographs of the new plant demonstrate the overall appearance of the plant including the unique traits. The plant in the photograph is of a one-year-old vernalized plant at a wholesale perennial nursery in Zeeland, Mich., USA. The colors are as accurate as reasonably possible with color reproductions. Some slight variation of color may occur as a result of lighting quality, intensity, wavelength, and direction or reflection.

FIG. 1 shows a plant in a shaded outdoor trial garden in Zeeland, Mich. in late spring flowering.

FIG. 2 shows a close-up of the foliage in mid-season coloration in a greenhouse.

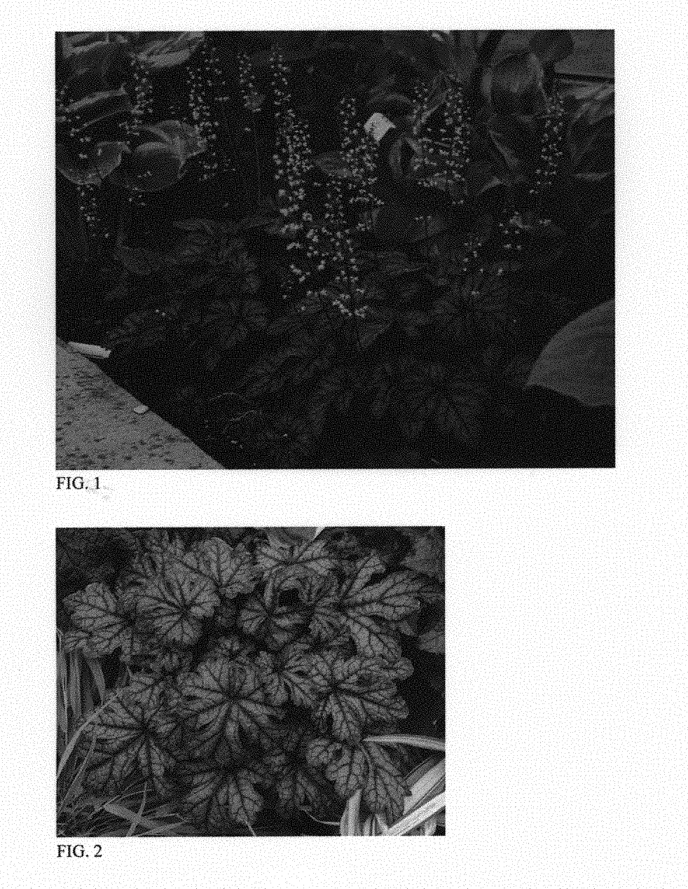

### DETAILED BOTANICAL DESCRIPTION

The following description is based on two-year-old plants growing in double poly greenhouse at a wholesale perennial nursery in Zeeland, Mich., USA. The new plant has not been grown under all possible environments and may phenotypically appear different under different conditions such as light, temperatures, fertilizer, and water, without any difference in genotype. The color descriptions are from the 2001 edition of The Royal Horticultural Society Colour Chart except where common dictionary terms are used.
Parentage: Unreleased proprietary Heuchera hybrid K10-70-69 with Heuchera ‘Mocha’ U.S. Plant Pat. No. 18,386 and Heuchera ‘PWHEU0109’ U.S. Plant Pat. No. 19,574 as the two grandparents on the female (seed parent); Tiarella ‘Jade Peacock’ as the male (pollen);
Plant habit: Hardy herbaceous perennial of tightly compact rhizomes with basal rosette of mounded foliage; foliage about 30 cm tall and about 52 cm in diameter;
Roots: Fibrous, finely branched; when actively growing near white in color depending on soil type;
Growth rate: Rapid, rooting from cutting in 2 weeks and finishing in three-liter container in about 3 months; beginning to flower in 6 to 8 weeks following a 9 week vernalization;
Foliage: Leaf blade puberulent adaxial and minutely puberulent abaxial; palmately five-lobed, apical lobes incised typically to nearly four-fifths of the way to petiole; matte surface above and slightly lustrous below; rounded lobe apices with apices and margin micro-ciliate, and cordate base with lower lobes imbricate to about 5.0 mm and apical lobe imbricate side lobes by about 1.0 to 2.0 mm; blade to about 12.0 cm long and 10.5 cm wide, average about 11.0 cm long and 9.5 cm wide; center lobe to about 6.0 cm long and 5.2 cm wide at widest point; average about 5.0 cm long and about 4.5 cm wide;
Foliage color: Leaf color is seasonally variable; young spring and expanding leaves adaxial nearest RHS 179A with margin portion nearest RHS 165B; young spring and expanding abaxial nearest RHS 185B; at flowering and mid-season adaxial nearest RHS N186A surrounding veins and nearest RHS 198B between veins; at flowering and mid-season abaxial nearest RHS N186C; winter leaves adaxial area closest to major veins blend between RHS N187A and RHS N189A, with a portion surrounding that closest to veins and surrounding minor veins nearest RHS 139A a lighter silvery green between the veins of between RHS 191B and RHS 191C;
Leaf margin: Crenate to ciliate;
Leaf apex: Rounded with spicule or minutely cuspidate;
Leaf base: Cordate with frequently imbricate basal lobes;
Leaf surface: Puberulent adaxial and minutely puberulent abaxial;
Leaf quantity: Dense, about ten to fifteen per division and 120 per plant;
Veins: Palmate, puberulent to glabrate adaxial and puberulent abaxial;
Vein color: Variable with season; adaxial expanding foliage nearest RHS 187B and abaxial blend between RHS 186B and RHS 186C; at time of flowering and mid-season adaxial nearest RHS 182B, at time of flowering and mid-season abaxial main veins blend between lighter than RHS 182D and RHS 191C; winter adaxial veins nearest RHS 182B and overwintered abaxial blend between RHS 182C and RHS 191B;
Petiole: Terete, pubescent, base clasping; to about 16.0 cm long and about 3.0 mm diameter at base; average about 14.5 cm long and 2.5 mm diameter at base; wiry but flexible;
Petiole color: On emerging foliage nearest RHS 186C with thin dark stripe running down adaxial center nearest RHS 186A; at flowering and mid-season nearest RHS 182C on abaxial side with thin adaxial strip of nearest RHS 187C; winter between RHS 148A and RHS 148B with thin strip nearest RHS N187A;
Inflorescence: In tightly-branched panicle, about 10 panicles per plant; about 230 flowers per panicle; first panicle flowering about the end of May in Michigan and continuing for four weeks; individual panicles remaining in flower for about three weeks; individual flowers open for about four days; flower attitude mostly outwards;
Fragrance: None detected;
Peduncle: Terete, erect; puberulent; to about 50.0 cm tall and about 3.0 mm diameter at base; flowering portion about 30.0 cm long and about 6.0 cm across;
Peduncle attitude: Upright erect;
Peduncle branches: Forty to fifty branches per panicle with two to twenty-four flowers per branch; extending between 15 to 30 degrees above horizontal; branches to about 2.5 cm long and about 0.7 mm diameter at base;
Peduncle color: Distal portion above foliage blend between RHS 187A and RHS 191A, basal portion between RHS 146B and RHS 146C;
Pedicel: Terete; puberulent; average about 2.5 mm long and 0.5 mm diameter at base;
Pedicel color: Nearest RHS 187A;
Buds one day prior to opening: Ellipsoid with rounded apex and rounded base; glandular to pubescent; about 3.0 mm long and 2.0 mm diameter;
Bud color: Mixture of both lighter than RHS 173D and RHS N186D;
Flower: Perfect, campanulate, actinomorphic; about 5.0 mm deep and 7.0 mm in diameter at face; individual flowers lasting about three to four days on plant or as cut panicles;
Flower attitude: Mostly outwardly;
Calyx: Typically five sepals; glandular abaxial, glabrous adaxial; apex rounded, fused at base into hypanthium; margin entire; sepals about 4.0 mm long and 1.5 mm wide at point of fusion;
Calyx color: Abaxial apex nearest RHS 148C with undertone of nearest RHS N186C, mid-portion and base lighter than RHS 155D with slight blush of RHS N186D; adaxial apex nearest RHS 146D and base lighter than RHS 145D;
Petals: Five; adnate to calyx; spatulate, acute apex, attenuate base; margin entire; glabrous abaxial and adaxial; about 4.0 mm long and 0.8 mm wide at widest point;
Petal color: Abaxial and adaxial nearest RHS N155B;
Androecium:
   Filaments.—Five, thin, about 2.5 mm long and less than 0.5 mm in diameter; color white, lighter than RHS N155D.
   Anthers.—Vestigial; basifixed; oblong to about 0.3 mm long and about 0.2 mm wide; color nearest RHS 155D.
   Pollen.—Not observed.
Gynoecium:
   Pistil.—One central two-beaked pistil.
   Style.—About 5.0 mm long and 0.2 mm at apex flaring to 2.0 mm at base; color apex white, lighter than RHS 155D and base nearest RHS 145D.
   Stigma.—Minute, about 0.2 mm diameter; color white, lighter than RHS 155D.
   Ovary.—Two carpels; ovoid, apex tapering to meet style; rounded base and sides; about 2.0 mm across at base and 2.5 mm tall; color nearest RHS 155D.
Fruit and seed: Sterile; not observed;
Disease and pest tolerance: X Heucherella ‘Galactica’ grows best with ample moisture and good drainage in part shade or protection from sun in the hottest part of the day. Cold hardy from USDA zones 4 to 9. X Heucherella ‘Galactica’ is able to tolerate heat and humidity better than many X Heucherella. Other pest and disease resistance and tolerance outside of that normal for X Heucherella is not known.

It is claimed:

1. The new and distinct ornamental plant named X Heucherella ‘Galactica’ as herein described and illustrated.
